(19) United States
(12) Patent Application Publication
(10) Pub. No.: US 2025/0178925 A1
(43) Pub. Date: Jun. 5, 2025

(54) WATER PURIFIER AND CONTROL METHOD THEREOF

(71) Applicant: LG ELECTRONICS INC., Seoul (KR)

(72) Inventors: Junseok YOU, Seoul (KR); Kyungsoo OH, Seoul (KR); Junsang YUN, Seoul (KR); Sangju LEE, Seoul (KR)

(21) Appl. No.: 18/904,790

(22) Filed: Oct. 2, 2024

(30) Foreign Application Priority Data
Dec. 5, 2023 (KR) 10-2023-0174162

(51) Int. Cl. C02F 1/00; C02F 1/02; E03C 1/044

(52) U.S. Cl. CPC C02F 1/008; C02F 1/003; C02F 1/02; E03C 1/044; C02F 2201/005; C02F 2209/02; C02F 2209/10; C02F 2307/10; E03C 2201/40

(57) ABSTRACT

A water purifier may discharge room-temperature water, cold water or hot water. The water purifier may comprise a hot water module configured to heat water; a cold water module configured to cool water; a sensing device disposed on a flow path in which water discharged from the hot water module and the cold water module flows; and a water discharge part which connects to the sensing device and from which water is discharged. The water purifier may measure water quality after adjusting the temperature of the sensing device to a temperature corresponding to a measurement standard temperature when the temperature of the sensing device differs from the measurement standard temperature after hot water or cold water is discharged.

### CROSS-REFERENCE TO RELATED APPLICATION(S)

This application claims priority to and the benefit of Korean Patent Application No. 10-2023-0174162 filed on Dec. 5, 2023, which is hereby incorporated by reference as if fully set forth herein.

### BACKGROUND

### 1. Field

The present disclosure relates to a water purifier and a control method thereof, and in particular, a water purifier and a control method thereof in which an error in an index value of measured water quality, caused by a difference in the temperature of water discharged and room temperature, may be avoided.

### 2. Background

Details in the background section do not constitute a prior art but are given only as background information concerning the subject matter of the present disclosure.

Water discharge devices may supply water or other liquids, and these devices may discharge a desired amount of water based on user manipulation or other input. For example, a user may specify a particular amount of water to be dispensed, or the user may position a container to receive the water and remove the container after a desired amount of the water is dispensed. The water discharge devices may be applied in a variety of fields and, in certain examples, may be included in appliances such as refrigerators, drink dispenser (e.g., a coffee or team maker) or water purifiers. A water discharge device provided in a refrigerator or a water purifier may be configured to supply a predetermined amount of water, based on user manipulation or other input. For example, the predetermined amount of water may be dispensed when the user positions a container to receive the water. In certain examples, water discharge devices may supply water of a desired temperature based on a user manipulation or other input. For example, the water discharge devices may selectively supply room-temperature water (or ambient temperature water), cold (or cooled) water, and hot (or heated) water.

In certain examples, the water discharge devices may dispense purified water, such as water that is passed through a filter and/or a sterilizer. For example, a water purifier may be connected to a water supply source, such as a water tap or a tank filled by a user, to receive raw water, and the water purifier then may pass the raw water through a filter to remove floating materials and potentially harmful materials and the like included in the raw water and then discharge a desired amount of the purified water based on user manipulation or other control signal.

A variety of water purifiers that purify raw water, heat or cool the purified water, and then supply the purified hot water or cold water are commercially available on the market. For example, a water purifier is discussed in Korean Patent No. KR 10-2449624, which is incorporated by reference herein where appropriate for appropriate teachings of additional or alternative details, features and/or technical background. Additionally, water purifiers may have a relatively compact size such that the water purifiers may be placed in various places, such as on a kitchen counter top.

In a water purifier, the quality of drinking water supplied to the user may be managed thoroughly to ensure the safety and taste of the purified water. For example, the quality of drinking water may be monitored to determine when the filter needs to be cleaned or replaced. To this end, the water purifier may include a sensing device that measures one or more attributes of water quality. The sensing device may measure various values related to water quality, such as turbidity (e.g., clearness), acidity (e.g., pH), concentrations of residual chlorines, concentrations of total dissolved solids (TDS), concentrations of dissolved oxygen, and the like.

For example, a sensing device measuring a TDS value may include a sensing electrode that is positioned in water to measure a frequency, conductivity, or other sensed attribute of a current applied to the water, and the sensing device may covert the measured attribute to measure a TDS value or other attribute of water quality. However, a current frequency value or other attribute measured by the sensing device may vary depending on a temperature of the water. In certain examples, the current frequency value or other attribute may be corrected based on a measured temperature of water, such that a TDS or other water quality value having a reduced error may be obtained. However, even though a measurement value for the current frequency value may be corrected based on a temperature of the water, a measurement error may still occur due to significant possible water temperature differences. For example, a water purifier may cool or heat water, and accordingly, the temperature of discharged water may vary over relatively large temperature ranges, such as between water that is cooled close to a freezing point or heated close to a boiling point (e.g., from about 5° C. to 90° C.). When a TDS or other water quality value is measured in water having such a wide range of temperatures, an error may occur due to a temperature change, making it hard to obtain an accurate TDS or other water quality value even when a correction is made based on temperature change.

To obtain a more accurate TDS or other water quality value, the water quality value may be measured at a temperature where an error in measurement of the water quality value decreases while the reliability in measurement of the water quality value increases. For example, it may be desirable to measure the TDS or other water quality value at an ambient room temperature or at a temperature relatively close to room temperature (e.g., with 10° C. of room temperature) because a relationship for conversion between a measured current frequency and the TDS or other water quality value may be established at a set temperature, such as room temperature. However, the ambient temperature may vary depending on a situation, location (e.g., different geographic position), and environment (e.g., indoors or outdoors) where the water purifier is installed. To address this concern and to improve overall measurement accuracy, there is a growing demand for a water purifier and a control method thereof that may measure a TDS or other water quality value in a range of temperatures that are close to room temperature and help to obtain a TDS or other water quality value having less error and greater reliability.

### BRIEF DESCRIPTION OF THE DRAWINGS

The embodiments will be described in detail with reference to the following drawings in which like reference numerals refer to like elements wherein:

FIG. 1 is a view schematically showing an example of a water purifier according to an embodiment;

FIG. 2 is depicts an example of a sensing device of a water purifier according to an embodiment;

FIG. 3 is a view schematically showing an example of a structure of a water purifier according to an embodiment;

FIG. 4 is a flowchart describing the operation of a water purifier according to ab embodiment;

FIG. 5 is a flowchart for describing a control method of a water purifier when hot water is discharged; and

FIG. 6 is a flowchart for describing a control method of a water purifier when cold water is discharged.

### DETAILED DESCRIPTION

The below-described aspects, features and advantages are specifically described hereinafter with reference to accompanying drawings such that one having ordinary skill in the art to which the subject matter of the present disclosure pertains may embody the technical spirit of the disclosure easily. In the disclosure, detailed description of known technologies in relation to the subject matter of the disclosure is omitted if it is deemed to make the gist of the disclosure unnecessarily vague. Hereinafter, preferred embodiments according to the disclosure are specifically described with reference to the accompanying drawings. In the drawings, identical reference numerals may denote identical or similar components.

The terms “first”, “second” and the like are used herein only to distinguish one component from another component. Thus, the components are not to be limited by the terms. Certainly, a first component may be a second component, unless stated to the contrary.

Throughout the disclosure, each component may be provided a single one or a plurality of ones, unless stated to the contrary. In the disclosure, singular forms include plural forms as well, unless explicitly indicated otherwise. In the disclosure, the terms “comprise”, “comprised of” and the like do not imply necessarily including stated components or stated steps and imply excluding some of the stated components or stated steps or including additional components or additional steps. Throughout the disclosure, the terms “A and/or B” as used herein may denote A, B or A and B, and the terms “C to D” may denote C or greater and D or less, unless stated to the contrary.

Throughout the present disclosure, an “up-down direction” denotes the up-down direction of a water purifier in the state where the water purifier is installed to be used as usual. A “left-right direction” denotes a direction across the up-down direction, and a front-rear direction denotes a direction across both the up-down direction and the left-right direction. “Both lateral directions” or a “lateral direction” have the same meaning as the left-right direction, and the directional terms are used in a mixed manner in the present disclosure.

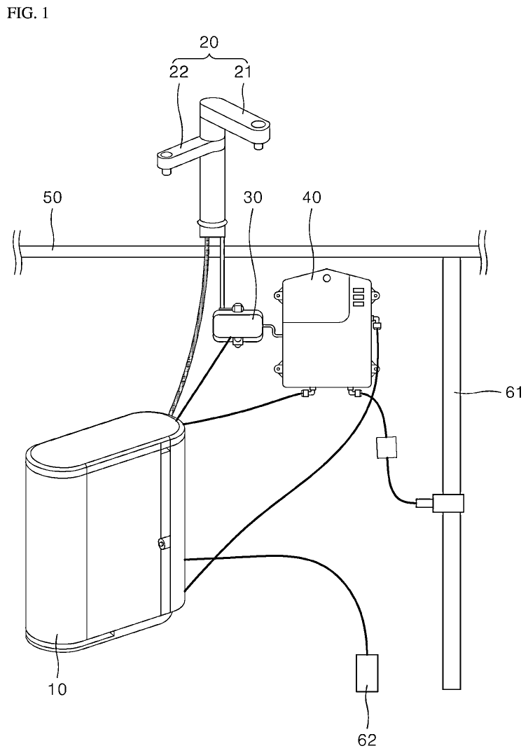

FIG. 1 is a view schematically showing aspects of a water purifier (or water purifier system) according to one embodiment. The water purifier may be installed in an indoor kitchen to filter water and may provide one or more of ambient-temperature water, hot water or cold water to the user. As used herein, “hot water” may refer to water or other liquid that is heated above an ambient temperature, and “cold water” may refer to water or other liquid that is cooled below the ambient temperature.

The water purifier may include a main body 10 that generates purified water that may be heated or cooled, and a water discharge part 20 (also referred to as tap or a faucet) fluidly connected to the main body 10 to output the purified water. The water discharge part 20 may be disposed to be exposed externally, such as at a sink 50 provided in the kitchen, such that the user may access and use the water discharge part 20 conveniently to discharge purified water received from the main body 10. Other components of the water purifier, such as the main body 10 and the like, may be disposed to not to be externally exposed, and may be positioned in a cabinet or a space below the sink 50, to ensure aesthetic qualities.

The main body 10 may be fluidly connected to a tap water pipe (or water source) 61 and may receive raw water from a well or a water supply via the tap water pipe 61. In another example, the man body may receive water from a tank or other component that stores water. Additionally, the main body 10 may be fluidly connected to a sewer pipe (or drain pipe) 62, and water stored in or passing through the main body 10 (e.g., from a drain part (or drain valve) 30) may be drawn out of the water purifier and discharged to the sewer pipe 62. Additionally, water in sink 50 (e.g., a portion of the water received from water discharge part 20) may be discharged toward the sewer pipe 62.

As depicted in FIG. 3, the main body 10 may include a filter part (or filter) 11 that filters the raw water, a hot water module (or heater) 12 to heat the filtered water, a cold water module (or cooler) 13 to cool the filtered water. Continuing with FIG. 1, the main body 10 or other component of the water purifier may include a controller, such as a control board in which a control unit or processor to control the operation of the water purifier may be embodied, and other components provided for the operation of the water purifier, such as a power source that drives the hot water module 12, the cold water module 13, and a sensing part (or sensing assembly) 40.

The water discharge part 20 may connect to the main body 10 through a pipe, and purified water from the main body 10 may be discharged from the water discharge part 20. Water discharged from main body 10 and through the water discharge part 20 may comprise, for example, drinking water to be drunk by the user, and wash water for the user to wash food, kitchenware and other things. The drinking water and the wash water may be water that is generated in the main body 10 by filtering raw water from tap water pipe 61. In certain implementations, the drinking water and the wash water may have different quality due to a different degree of filtration by the main body 10. For example, the drinking water may have a relatively greater degree of filtration (e.g., by being filtered for a greater duration or by passing through a relatively greater number of filters 11) compared to the wash water such that the user can safely drink the drinking water. When desired, the drinking water may be heated or cooled before being dispensed from water discharge part 20. The wash water to wash kitchenware, food and the like may be sterilized room-temperature water dispensed from water discharge part 20. In other examples, the wash water may be heated or cooled before being dispensed from water discharge part 20.

In the example described above in which the drinking water and the wash water have different quality, the drinking water and the wash water are generally not mixed. Thus, the drinking water and the wash water may flow along separate flow paths through the water discharge part 20. For example, the water discharge part 20 may include separate flow paths through which the drinking water and the wash water respectively flow. For example, the water discharge part 20 may include a drinking water discharge opening (or also referred to as a first or drinking water outlet) 21 through which drinking water is discharged, and a wash water discharge opening (also referred to as a second or wash water spout) 22 through which wash water is discharged, respectively.

In certain examples, to allow the wash water and the drinking water to flow separately, the wash water may flow through a wash water pipe that directly connects the main body 10 and the water discharge part 20 such that the wash water can be discharged from the water discharge part 20. Thus, the wash water may flow through the wash water pipe from the main body 10 to the water discharge part 20 without passing through a sensor (e.g., sensing part 40) or drain part 30.

However, the quality of the drinking water may be monitored and managed since the user drinks the drinking water. For example, the drinking water discharged from the main body 10 may be discharged toward the water discharge part 20 through a fluid path that includes a sensing part (or sensor assembly) 40 and a drain part 30 described hereinafter that connect between the main body 10 and the water discharge part 20 through at least one pipe. For example, the drain part 30 may be fluidly connect to the main body 10 and the water discharge part 20 through at least one pipe. The sensing part 40 may be fluidly connected to the main body 10 and the drain part 30 through at least one pipe, and the sensing part 40 may sense a water quality of drinking water to be dispensed through the water discharge part 20. Thus, the sensing part 40 and the drain part 30 may be disposed between the main body 10 and the water discharge part 20 on a flow path in which the drinking water flows. For example, the sensing part 40 may directly connect to the main body 10 such that drinking water discharged from the main body 10 may be drawn into the sensing part 40. Further, the drain part 30 may directly connect to the water discharge part 20, and directly connect to the main body 10.

Water drawn into the drain part 30 may selectively flow to the water discharge part 20 or the main body 10. If water drawn into the drain part 30 is to be drunken by the user, the drain part 30 may guide the water to flow to the water discharge part 20 to be discharged to the user. Alternatively, if water drawn into the drain part 30 is not for the user to drink, the water may flow to the main body 10 from the drain part 30 and be drained to the sewer pipe 62 from the main body 10. To this end, the drain part 30 may include a component, such as a diverter valve (e.g., second diverter valve 31 in FIG. 3), to adjust a flow direction of water between either the water discharge part 20 or the main body 10.

In certain examples, water may flow in the water purifier in the following manner. For example, raw water in the tap water pipe 61 may pass through the sensing part 40 and may be drawn again into the main body 10. Wash water that is generated by sterilizing and filtering raw water in the main body 10 may flow directly to the water discharge part 20 and be discharged from the wash water discharge opening 22 of the water discharge part 20. Additionally, drinking water that is generated by filtering raw water and then optionally heated or cooled in the main body 10 may be drawn into and pass through the sensing part 40, pass through the drain part 30, and is then discharged from the drinking water discharge opening 21 of the water discharge part 20. Additionally, the drain part 30 may direct at least some of the drinking water back to the main body 10 (e.g., when the water is not of sufficient quality to be drunk) to be drained to the sewer pipe 62, when desired.

When drinking water is formed in the main body 10 by filtering the raw water, the drinking water may pass through the sensing part 40 repeatedly such that the quality of the drinking water may be managed thoroughly. For example, while raw water (e.g., the drinking water in a raw-water state before being filtered and treated within the main body 10) passes through the sensing part 40, the quality of the raw-state drinking water may be measured by the sensing part 40, and while the drinking water in a drinking water state (e.g., after the raw water is filtered and treated within the main body 10) passes through the sensing part 40 again, the quality of the drinking water may be again measured by the sensing part 40, such as to determine improvements in one or more measured water quality factors.

The sensing part 40 may measure the quality of raw water and drinking water. For example, the sensing part 40 may comprise at least one of a turbidity sensor, an ion sensor, a chlorine sensor, a total dissolved solids (TDS) sensor, a biochemical oxygen demand (BOD) sensor, or other water quality sensor. The sensing part 40 may measure at least one index value, e.g., at least one of turbidity, pH, residual chlorines, TDS, and dissolved oxygen, of drawn water. Accordingly, the sensing part 40 may include one or more sensing devices (or sensors) 41 disposed at the sensing part 40, and the one or more sensing devices 41 may measure a variety of index values depending on the configurations and types of the one or more sensing devices 41 included in the sensing part 40.

Hereinafter, a sensing device 41 comprising a TDS sensor sensing the quantity of total dissolved solids (TDS) included in water and a temperature of the water is described specifically. It should be appreciated, however, that the sensing part 40 may include a variety of other sensors that measure one or more other types of index values different from or in addition to a TDS value.

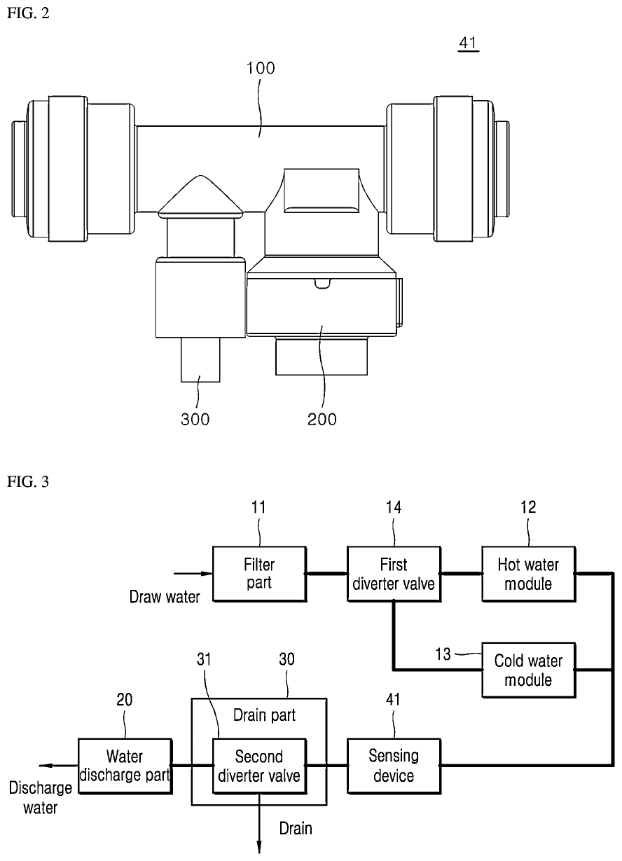

FIG. 2 is a view schematically showing a sensing device 41 according to one embodiment. The sensing device 41 may be provided at the sensing part 40, and may be disposed on a flow path in which water flows to measure the temperature and quality of the water. Water quality may be measured, for example, based on a TDS value or other water quality value. To measure an accurate TDS value, the water purifier of in the depicted example may also measure temperature, considering the characteristics of a TDS value may vary depending on temperature. Accordingly, the temperature of water may be measured, and based on the temperature of the water, a TDS value may be determined more accurately.

A water quality measurement point (e.g., a location where the water quality value is measured) and a temperature measurement point (e.g., a location where a temperature of the water is measured) may differ, but it is preferable that the quality measurement point be as close as possible to the temperature measurement point. The temperature of water may vary at different positions in a flow path. For example, hot water may cool when travelling away from hot water module 12. When the quality measurement point and the temperature measurement point are spaced apart from each other, the temperature of the water at the quality measurement point may not be measured accurately. To prevent this from happening, it may be preferable to provide one sensing device 41 that measures both the temperature and quality of water at relatively close locations.

The sensing device 41 may comprise a body 100, a temperature sensing part (or temperature sensor) 300, and a quality sensing part (or water quality sensor) 200. The body 100 may have a space which fluidly connects to and receives water from an external pipe through which the water flows, and the received water may flow through the space of body 100. The temperature sensing part 300 and the quality sensing part 200 may be disposed in the lengthwise direction of the body 100, e.g., in a direction across the direction in which water flows.

The temperature sensing part 300 may be disposed at the body 100 and measure the temperature of water. A sensing portion (e.g., probe) of the temperature sensing part 300 may sink into or otherwise contact water in a flow path formed in the body 100.

The quality sensing part 200 may be disposed at the body 100 in such a way that the quality sensing part 200 is separated from the temperature sensing part 300 and measures an aspect of water quality. Similarly, a sensing portion (e.g., sensor probe) of the quality sensing part 200 may sink in or otherwise contact water in the flow path formed in the body 100. The quality sensing part 200 may comprise a TDS sensor sensing the quantity of total dissolved solids (TDS) included in water, and a measurement value in relation to water quality may include a TDS value. It should be appreciated that quality sensing part 200 may additionally or alternatively sense other aspects of water quality.

At the sensing device 41, the temperature sensing part 300 and the quality sensing part 200 may be separate from each other but may be integrally provided to the sensing device 41 to be disposed such that the temperature measurement point and the quality measurement point may be relatively close to each other (e.g., separated by less than 10 cm). Accordingly, since a temperature and a TDS value of water may be measured by sensing device 41 at positions that are substantially adjacent to each other, to the extent that a measurement error may be minimized, an accurate TDS value based on a temperature may be applied in relation to the operation of the water purifier that is described hereinafter. Thus, the temperature measurement point where the temperature is measured and the quality measurement point where the water quality is measured may be substantially identical, and hereinafter, the temperature measurement point and the quality measurement point may be collectively referred to as a “measurement point” without distinguishing from each other.

Aspects of a water purifier according to certain examples are depicted in FIG. 3. As a device relating to the flow of water, the water purifier may comprise filter (or filter) part 11, hot water module 12, cold water module 13, and a first diverter valve 14 that are disposed in the main body 10. As illustrated in FIG. 3, each of the components of the water purifier may connect to one another in series or in parallel through at least one pipe. The sensing device 41 may be disposed downstream of the hot water module 12 and the cold water module 13, and may be disposed on a flow path in which water discharged from at least one of the hot water module 12 and the cold water module 13 flows to measure the temperature and quality of the water outputted from the hot water module 12 and the cold water module 13.

Raw water (e.g., from tap water pipe 61) may pass through the filter part 11 and become filtered room-temperature water. The hot water module 12 and the cold water module 13 may be disposed between the filter part 11 and the sensing device 41 to receive the filtered room-temperature water. In certain examples, the hot water module 12 and the cold water module 13 may be provided in parallel along separate water paths between the filter part 11 and the sensing device 41.

The hot water module 12 may heat water and change the filtered room-temperature water to hot (or heated) water. For example, the hot water module 12 may include an electric heating device that receive electricity from the outside and converts the electricity to heat flowing water. For example, the electric heating device may include a heating coil, an induction heater, a thermoelectric element (e.g., a Peltier device), etc.

The cold water module 13 may cool water and change the water to cold water. The cold water module 13 may cool water based on, for example, a refrigeration cycle using a compressed refrigerant, a cooling system using a thermoelectric element (e.g., a Peltier device), and the like. Water having passed through the hot water module 12 or the cold water module 13 may be drawn into the sensing device 41, and the sensing device 41 may measure the temperature and quality of the water.

In certain implementations, the first diverter valve 14 may be disposed between the filter part 11 and the hot water module 12 and the cold water module 13. The first diverter valve 14 may operate according to an instruction of the control unit controlling the operation of the water purifier, to manage a flow direction of room temperature filtered water to at least one of the hot water module 12 or the cold water module 13.

Additionally, the water discharge part 20 may connect to and receive water from the sensing device 41, and the received water may be discharged from the water discharge part 20. For example, room-temperature water, hot water, cold water, or a combination thereof may be discharged from the water discharge part 20. For example, the first diverter valve 14 may operate to guide the room temperature filtered water from filter part 11 to pass through the hot water module 12, but when the hot water module 12 does not operate, the room-temperature water is not heated and may be discharged to the water discharge part 20. When the hot water module 12 does operate, the room-temperature water received in the hot water module 12 is heated and may be discharged to the water discharge part 20. In another example, the first diverter valve 14 may operate to guide the room temperature filtered water from filter part 11 to pass through the cold water module 13, but when the cold water module 13 does not operate, the room-temperature water is not cooled and may be discharged to the water discharge part 20. When the cold water module 13 does operate, the room-temperature water received in the cold water module 13 is cooled and may be discharged to the water discharge part 20.

The water purifier may also include drain part 30, and the drain part 30 may be positioned between the sensing device 41 and the water discharge part 20, such as to receive at least a portion of water output from the sensing device 41. Further, the water purifier may comprise a second diverter valve 31, and the second diverter valve 31 may be disposed in the drain part 30. The second diverter valve 31 may operate according to an instruction of the control unit and change a flow direction of water discharged from sensing device 41 to at least one of the water discharge part 20 or to the outside of the water purifier (e.g., to the sewer pipe 62) and away from water discharge part 20. Accordingly, the second diverter valve 31 may direct water from sensing device 41 that is to be drunken by the user to the water discharge part 20, and may direct water from sensing device 41 that is not to be drunken by the user or used for washing to be drained outward from the water purifier through the drain part 30.

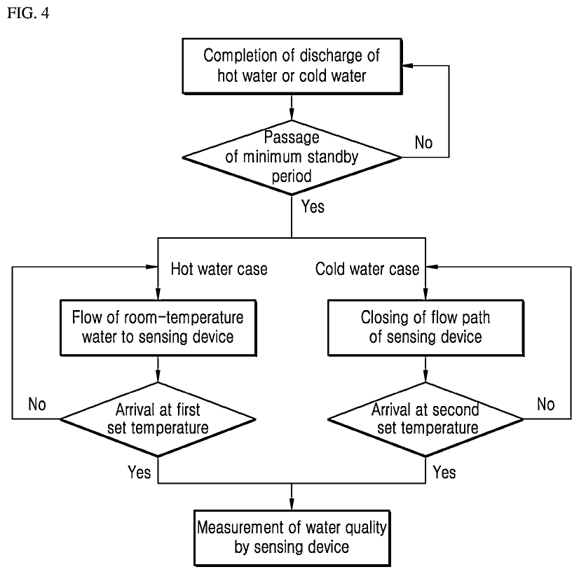

FIG. 4 is a flowchart describing the operation of a water purifier in one embodiment. As previously described, a water quality sensor 200 may measure a characteristic, such as change in frequency or voltage of a current applied to the water, an a TDS value associated with this measured characteristic may vary depending on temperature. A TDS value measured by the quality sensing part 200 may be a value that is set at room-temperature (e.g., respective TDS values are associated with respective characteristic measurements). When the temperature of water is not a set temperature (e.g., a room temperature), there may be an error in the TDS value associated with the measured characteristic value. Herein, the set temperature at room temperature may be 20 or 25° C., for example. Hereinafter, the set temperature at room temperature may be referred to as a “measurement standard temperature”.

Since water passing through the sensing device 41 may be greater or less than the measurement standard temperature based on the operation of the hot water module 12 or the cold water module 13 or due to an ambient temperature of the water being different from the measurement standard temperature (e.g., cooler in winter and warmer in summer), there may be an error in the TDS value associated with the measured water characteristic value. To reduce the error, the temperature in the sensing device 41 and the quality measurement point should match the measurement standard temperature or be at a temperature similar to the measurement standard temperature (e.g., whether an acceptable error range, such as with ±5° C. of the measurement standard temperature).

After hot water or cold water flows into the sensing device 41, temperature at the measurement point may not be the measurement standard temperature because of a heat transfer between the hot water or the cold water flowing in the sensing device 41 and the wall of the sensing device 41, which is heated or cooled. Subsequently, the heated or cooled sensing device 41 may not accurately measure the TDS value. To address this concern, the water purifier according to certain embodiments may operate such that a temperature at the measurement point is adjusted to be at or similar to (e.g., within a preset temperature range of) the measurement standard temperature, such that the occurrence of an error caused by cooling or heating may be reduced after hot water or cold water flows into the sensing device 41.

When room-temperature water that is not heated or cooled is discharged from the sensing device 41 and through the water discharge part 20, an accurate TDS value may be obtained in the sensing device 41 since temperature at the measurement point in the sensing device 41 may be at or closely correspond to the measurement standard temperature. Thus, after room-temperature water is dispensed, the TDS value may be accurately measured by the sensing device 41 without an additional process or an additional action to adjust the temperature at the sensing device 41.

When hot water or cold water is discharged from the water discharge part 20 after passing through the sensing device 41, the water purifier may be controlled, as described hereinafter, to operate to adjust a temperature of the sensing device 41 to be at or similar to the measurement standard temperature. For example, as described below, the water purifier may operate to adjust the temperature of the sensing device 41 to correspond to the measurement standard temperature.

In certain implementations, the operation of the water purifier may not be adjusted while the user is using the water purifier, so as to not to cause inconvenience to the user (e.g., by changing the temperature of dispense water from a requested temperature). For example, when the user does not use the water purifier for a set minimum standby period, it may be expected that the user is no longer using the water purifier, and the temperature of the sensing device 41 may be adjusted such that water quality is more accurately measured. The minimum standby period may be, for example, 2 minutes from the time point when hot water or cold water is last discharged from the water discharge part 20 completely, but the minimum standby period is not limited to this specific time and may be set to any other time.

As described below, a method of adjusting a temperature of the sensing device 41 when hot water is discharged from the water discharge part 20 may differ versus a method of adjusting the temperature of the sensing device 41 when cold water is discharged from the water discharge part 20. The different methods are described separately based on each of the cases.

When hot water is discharged from the water discharge part 20, the temperature at the quality measurement point in the sensing device 41 may be greater than the measurement standard temperature. In this situation, the water purifier may operate to cool the sensing device 41 such that the temperature at a quality measurement point may be reduced to the measurement standard temperature or a temperature similar to the measurement standard temperature.

When hot water is discharged from water discharge part 20, the sensing device 41 and a pipe connecting to the sensing device 41 may be at a temperature greater than the measurement standard temperature. To cool the sensing part 41 after hot water is discharged from the water discharge part 20, room-temperature water may be allowed to flow to the sensing device 41. For example, room-temperature water may be allowed to flow to the sensing device 41 following a predetermined minimum standby period later after the hot water is discharged completely (e.g., the water discharge part 20 is turned off). As used herein, the room-temperature water may refer to ambient-temperature water that is not heated or cooled by water heating module 12 or water cooling module 13, and it should be appreciated that the term “room-temperature” water may be relatively warmer or cooler than air temperature within a space where the purifier is located. In other examples, the room-temperature water may be heated by hot water module 13 or cooled by cold water module to be at a set temperature (e.g., at a temperature within a space where the purifier is located) before being provided to the sensing device.

In this example in which sensing device 41 is cooled after hot water is discharged from water discharge part 20, the sensing device 41 that is heated by the hot water to have a temperature greater than the measurement standard temperature may be cooled rapidly by room-temperature water. Accordingly, temperature of a cooling device may rapidly return to a temperature the same as or similar to the measurement standard temperature and obtain an accurate measurement value of water quality.

In certain implementations, the room-temperature water may be allowed to flow to the sensing device 41 until the temperature at the measurement point of the sensing device 41 becomes a first set temperature that differs from the measurement standard temperature. The first set temperature, to which the sensing device 41 is cooled after hot water is dispensed, may correspond to a temperature at which the TDS value may be reliably measured by the sensing device 41. For example, the sensing device 41 may measure the TDS value with no error or a little error (e.g., within a present measurement amount) with respect to the measurement temperature at the first set temperature. After the temperature at the measurement point of the sensing device 41 reaches the first set temperature, the sensing device 41 may measure the TDS value or other water quality value.

When cold water is discharged from the water discharge part 20, the sensing device 41 and the pipe connected to the sensing device 41 may be cooled from a heat exchange with the cold water such that a temperature at the quality measurement point in the sensing device 41 may be less than the measurement standard temperature. At this time, the temperature of the sensing device 41 may be increased such that the temperature of the sensing device 41 reaches the measurement standard temperature or a temperature similar to the measurement standard temperature to improve measurement accuracy. For example, after cold water is discharged from the water discharge part 20, the flow path of the sensing device 41 may be closed such that no further water (cold, hot, or room temperature water) flows through and effects a temperature of the sensing device 41 until a temperature at the measurement point of the sensing device 41 reaches a desired temperature (e.g., a second set temperature to be discussed below). For example, the flow path of the sensing device 41 may be closed during a minimum standby period later after the cold water is discharged completely.

In one example, when hot water is discharged, the sensing device 41 may have a temperature much greater than room temperature, e.g., 80° C. or greater, due to a heat exchange with the hot water. For example, there may be a relatively large heat exchange between the sensing device 41 and the hot water since the hot water tends to be significantly warmer than room temperature. Accordingly, when hot water is discharged, room-temperature water is allowed to flow to the sensing device 41 for cooling since the sensing device 41 would be relatively hot and should be cooled by a relatively large amount before returning to room temperature. However, when cold water is discharged, the temperature of the sensing device 41 is cooled by a heat exchange with the cold water, but there may be a relatively small heat exchange between the sensing device 41 and the cold water since the cold water tends to be slightly cooler than room temperature. A difference between the temperature of the sensing device 41 and room temperature may be 5° C. or greater, for example. Accordingly, room-temperature water does not need to flow to the sensing device 41, to increase the temperature of the sensing device 41 to an acceptable level for accurate measurement, and it is possible to increase the temperature of the sensing device 41 naturally based on a heat transfer with the surroundings environment without directing room temperature or hot water to the sensing device 41, thereby achieving energy savings and greater ease of use.

Thus, after the cold water is discharged, the flow path of the sensing device 41 is cooled and has a temperature less than the measurement standard temperature may be closed, such that the temperature of the sensing device 41 may increase naturally. For example, the control unit may operate at least one of the first diverter valve 14 and/or the second diverter valve 31 and close the flow path of the sensing device 41 until the temperature of the sensing device 41 returns to a desired temperature of a second set temperature due to ambient warming. Like the first set temperature, the second set temperature may be a temperature ensuring the reliability of a measured TDS value, and the sensing device 41 at the first set temperature may have no error or a little error with respect to the measurement temperature. As temperature at the measurement point of the sensing device 41 reaches the second set temperature, the sensing device 41 may measure a water quality, e.g., a TDS value. Accordingly, the temperature of the cooling device may return to a temperature the same as or similar to the measurement standard temperature and obtain an accurate measurement of water quality, and the sensing device 41 may not be forcibly heated to increase its temperature, ensuring energy savings.

In one embodiment, the first set temperature and the second set temperature may be the same. For example, the first set temperature and the second set temperature may be set to the measurement standard temperature, e.g., 25° C. At this time, since there is no error in a TDS value caused by a difference between temperature at the measurement point and the measurement standard temperature, an accurate TDS value may be measured.

However, it may take a significant amount of time for the temperature of the sensing device 41 to reach the measurement standard time. Additionally, since the temperature of the sensing device may be a temperature of a place where the water purifier is disposed at room temperature, the temperature of the sensing device may vary depending on an environment and a season.

Thus, the first set temperature and the second set temperature may not reach the measurement standard temperature. For example, when the measurement standard temperature is 25° C. and room temperature around the water purifier is different from the measurement standard temperature (e.g., 22° C., 28° C. and the like), the first set temperature and the second set temperature may not reach the measurement standard temperature due to ambient heating after supplying cold water or cooling from water at room temperature after supplying hot water.

Considering these factors, the first set temperature (to which the sensing device 41 is cooled after hot water is dispensed) may be set to a temperature greater than the second set temperature (to which the sensing device 41 is warmed after cold water is dispensed), in another embodiment. Additionally, the first set temperature may be greater than the measurement standard temperature, and the second set temperature may be less than the measurement standard temperature.

For example, when the measurement standard temperature is 25° C., the first set temperature may be set to 30° C., and the second set temperature may be set to 20° C. It should be appreciated that these values are provided merely as examples, and the first and second set temperatures may be set to different values. The first set temperature and the second set temperature slightly differ from the measurement standard temperature (e.g., 5° C., in this example), but since the difference is small, an error in a sensed TDS value, caused by the difference in the temperature of the sensing device 41 and the measurement standard temperature, may be ignored.

Further, the first set temperature may be set to a temperature greater than 30° C., and the second set temperature may be set to a temperature less than 20° C., in another example. In this example, an error in a TDS value, as caused by the relatively greater difference in the temperature of the sensing device 41 and the measurement standard temperature, may increase, but a TDS value may be measured rapidly.

In the embodiment, when an error in the measurement value of water quality occurs due to a difference in the measurement standard temperature and an actual temperature of water as hot water or cold water is discharged to the water purifier, the temperature of the sensing device 41 may be adjusted to room temperature the same as or similar to the measurement standard temperature, to measure water quality. Accordingly, an error of a water quality value detected by sensing device 41 and caused by a temperature difference may be resolved, and a more accurate measurement value may be obtained.

As described above, the water purifier may comprise a drain part 30 that is disposed between the sensing device 41 and the water discharge part 20 and that drains water outward. As previously described, room-temperature water may be supplied to the sensing device 41 to cool the sensing device 41 after hot water is discharged, and this room-temperature water flowing for cooling in the sensing device 41 may be drained outward through the drain part 30. Thus, the room-temperature water flowing for cooling the sensing device 41 is not dispensed through the water discharge part 20.

For example, the room-temperature water flowing to the sensing device 41 may be drawn into the drain part 30 and drained outward (e.g., through sewer pipe 62), a minimum standby period after the hot water is discharged from the water discharge part 20 completely. Since after the minimum standby period passes, water flowing to cool the sensing device 41 is not water that is drunken and desired by the user, it is proper to drain water flowing to the sensing device 41 for a cooling period.

As the temperature at the measurement point of the sensing device 41 reaches the first set temperature due to the flow of cooling water after the hot water is discharged from the water discharge part 20, the sensing device 41 may measure water quality in the state in which room-temperature water is flowing in the sensing device 41 and is drained outward through the drain part 30. For example, as the temperature of the sensing device 41 reaches the first set temperature after the sensing device 41 is cooled due to a first amount of the room-temperature water flowing to the sensing device 41, a second, additional amount of room-temperature water continues to flow to the sensing device 41, and the sensing device 41 may measures water quality of the second amount of room-temperature water. At this time, the second amount of room-temperature water flowing to the sensing device 41 and evaluated to measure water quality may not be drunken by or desired by the user. Thus, the sensed water may be drained outward from the drain part 30 and not supplied to the water discharge part 20.

As previously described, room-temperature water may not be supplied to the sensing device 41 to warm the sensing device 41 after cold water is discharged. Thus, since the sensing device 41 may be closed and may not receive water during a warming period after cold water is discharged, room-temperature water may not flow to the sensing device 41 during the warming period, and the sensing device 41 is unable to measure water quality when temperature at the measurement point of the sensing device reaches the second set temperature due to ambient. Accordingly, as temperature at the measurement point of the sensing device 41 reaches the second set temperature after cold water is discharged from the water discharge part 20, the water purifier may supply room-temperature to the sensing device 41, and the sensing device 41 may measure water quality in the state when room-temperature water flows in the sensing device 41. This sensed water may be drained outward through the drain part 30 and not supplied to the water discharge part 20 since the user did not request the room-temperature water.

Thus, when water quality is measured after hot water or cold water is discharged and the temperature of the sensing device 41 returns to a temperature the same as or similar to the measurement standard temperature (e.g., to the first or second set temperature) as described above, room temperature water flows to the sensing device 41 without being heated or cooled for evaluation so that the temperature of the sensing device 41 is not significantly changed. At this time, the water purifier may stop the hot water module 12 and the cold water module 13 from operating while the sensing device 41 measures water quality to prevent the sensing device 41 from being changed during measurements.

Next, described is a control method of a water purifier that measures water quality by using a sensing device 41 after hot water or cold water is discharged from a water discharge part 20 completely. Hereinafter, particulars described above in relation to the control method of a water purifier may not be repeated. The water purifier may be controlled under the control of a control unit that is provided at the water purifier.

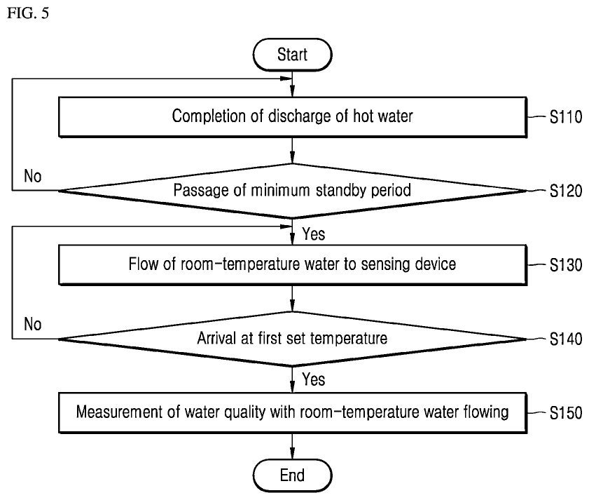

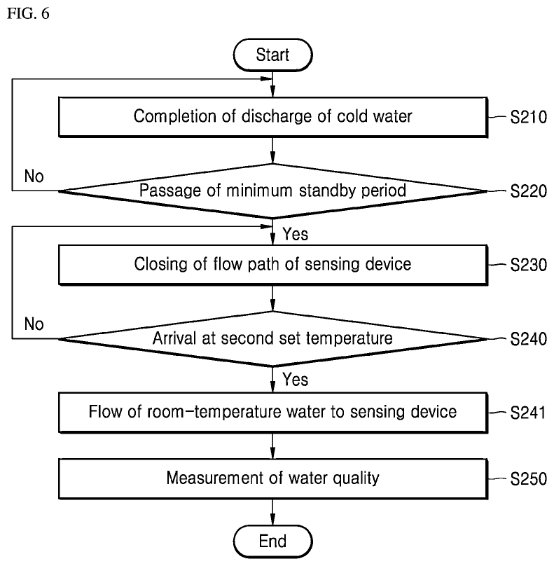

FIG. 5 is a flowchart for describing a control method of a water purifier when hot water is discharged from a water discharge part 20. FIG. 5 shows a control method of a water purifier for obtaining an accurate measurement value of water quality after hot water is discharged from the water purifier. For example, when hot water is discharged, the water purifier may complete the discharge of the hot water (S110). For example, as the user manipulates the water purifier to provide an input or other request for hot water, hot water may be discharged. The discharge of cold water, as described hereinafter with respect to FIG. 6, may be similarly performed in the same way as the user manipulates the water purifier to provide an input or other request for cold water.

The control unit may determine whether a set minimum standby time has passed after the hot water is discharged completely (S120). After the minimum standby period passes (S120-Yes), the control unit may allow room-temperature water to flow to a sensing device 41 (S130), and cool the sensing device 41 heated to have a temperature the same as or similar to a measurement standard temperature. As previously described, in S120, first diverter valve 14 may operate to direct room-temperature filtered water to hot water module 12 while the hot water module 12 is inactive to pass the filtered water without heating toward sensing device 41.

The control unit may check whether a temperature at a measurement point of the sensing device 41 reaches a first set temperature (S140). When the temperature at the measurement point of the sensing device 41 reaches the first set temperature, the sensing device 41 may measure water quality (S150). For example, as previously described, after the temperature at a measurement point of the sensing device 41 reaches the first set temperature (S140-Yes), an additional amount of room-temperature water may be supplied to the sensing device 41, and the sensing device 41 may measure water quality of this additional water.

As previously described, the water purifier may comprise a drain part 30 that is disposed between the sensing device 41 and the water discharge part 20 and drains water outward. In steps S130 and S150, room-temperature water flowing to the sensing device 41 may be drawn into the drain part 30 and drained outward following a minimum standby period later after hot water is discharged from the water discharge part 20 completely in step S110. Then, as the temperature at the measurement point of the sensing device 41 reaches the first set temperature after hot water is discharged from the water discharge part 20 completely (S140-yet), the sensing device 41 may measure water quality in the state where room-temperature water flows in the sensing device 41 and is drained outward through the drain part 30 (S150).

In the above-described process depicted in FIG. 5, after hot water is discharged, room-temperature water flows to the sensing device 41, a temperature at the measurement point of the sensing device 41 becomes a temperature that is the same as or similar to the measurement standard temperature (e.g., a first set temperature), and then water quality is measured to obtain an accurate measurement value.

FIG. 6 is a flowchart for describing a control method of a water purifier after cold water is discharged from a water discharge part 20. FIG. 6 shows a control method of a water purifier for obtaining an accurate measurement value of water quality when cold water is discharged from the water purifier.

When cold water is discharged, the water purifier may complete the discharge of the cold water (S210) during which sensing device 41 is cooled due to a heat exchange with the cold water. After the cold water is discharged completely, the control unit may check whether a set minimum standby period has passed after the cold water was discharged (S220).

After the minimum standby period passes (S220-Yes), the control unit of the water purifier may operate at least one of a first diverter valve 14 and/or a second diverter valve 31 to close the flow path of the sensing device 41 (S230) to prevent water from following to the sensing device 41 so that the sensing device may warm due to a heat exchange with ambient air. The control unit may check whether temperature at the measurement point of the sensing device 41 reaches a second set temperature (S240).

As the temperature reaches the second set temperature (S240-Yes), the control unit of the water purifier may operate at least one of the first diverter valve 14 and/or the second diverter valve 31 again to allow room-temperature water to flow to the sensing device 41 (S241). The sensing device 41 may then measure water quality in the state where the room-temperature water flows (S250).

Thus, after cold water is discharged, water flow is stopped to sensing device 41 to allow the temperature at the measurement point of the sensing device 41 to rise from being warmed by the ambient environment, and after the temperature reaches the second set temperature after the cold water is discharged from the water discharge part 20, water flow may resume to the sensing device 41, and the sensing device 41 may measure water quality in the state where room-temperature water flows in the sensing device 41 and is drained outward through the drain part 30.

In the above-described process, the temperature of the sensing device 41 increases naturally, temperature at the measurement point of the sensing device 41 becomes a temperature the same as or similar to the measurement standard temperature (e.g., the second set temperature), and then water quality is measured to obtain an accurate measurement value after cold water is discharged.

An aspect of the present disclosure is to provide a water purifier and a control method thereof that has a structure in which an error in an index value of a measured water quality, caused by a difference in the temperature of water discharged at a temperature different from room temperature, may be avoided, to ensure an accurate measurement of the index value. The aspect of the present disclosure is to provide a water purifier and a control method thereof that has a structure in which an accurate index value of water quality may be obtained when cold water is discharged. An aspect of the present disclosure is to provide a water purifier and a control method thereof that has a structure in which an accurate index value of water quality may be obtained when cold water is discharged.

Aspects according to the present disclosure are not limited to the above ones, and other aspects and advantages that are not mentioned above may be clearly understood from the following description and may be more clearly understood from the embodiments set forth herein. Additionally, the aspects and advantages in the present disclosure may be realized via means and combinations thereof that are described in the appended claims.

A water purifier of one embodiment may be provided to discharge room-temperature water, cold water or hot water. The water purifier may comprise a hot water module configured to heat water; a cold water module configured to cool water; a sensing device disposed on a flow path in which water discharged from the hot water module and the cold water module flows; and a water discharge part which connects to the sensing device and from which water is discharged. The water purifier may measure water quality after adjusting the temperature of the sensing device to a temperature the same as or similar to a measurement standard temperature when the temperature of the sensing device differs from the measurement standard temperature due to hot water or cold water being discharged.

When hot water is discharged from the water discharge part, the water purifier may allow room-temperature water to flow to the sensing device following a set minimum standby period after the hot water is discharged completely such that temperature at a measurement point of the sensing device cools to become a first set temperature, and after the temperature reaches the first set temperature, the sensing device may measure water quality.

Additionally, when cold water is discharged from the water discharge part, the water purifier may close a flow path of the sensing device following a minimum standby period later after the cold water is discharged completely such that a temperature at the measurement point of the sensing device warms from ambient heat to become a second set temperature, and as the temperature reaches the second set temperature, the sensing device may measure water quality.

A control method of a water purifier may involve measuring water quality by using the sensing device after hot water or cold water is discharged from the water discharge part completely. In a case where the hot water is discharged, a control method of a water purifier may comprise completing discharge of hot water; checking whether a passage of a set minimum standby period after the discharge of the hot water is completed; allowing room-temperature water to flow to the sensing device after the set minimum standby period; checking whether a temperature at a measurement point of the sensing device reaches a first set temperature; and measuring water quality by the sensing device after the temperature at the measurement point of the sensing device reaches the first set temperature.

In a case where the cold water is discharged, a control method of a water purifier may comprise completing discharge of cold water; checking whether a passage of a set minimum standby period after the discharge of the cold water is completed; closing a flow path of the sensing device after the set minimum standby period; checking whether temperature at a measurement point of the sensing device reaches a second set temperature after the flow path of the sensing device is closed; and reopening the flow path and measuring water quality by the sensing device after the temperature at the measurement point of the sensing device reaches the second set temperature.

The water purifier and the control method thereof according to the present disclosure may involve adjusting the temperature of the sensing device to room temperature to be the same as or similar to a measurement standard temperature and measuring water quality, when an error in a measurement value of water quality occurs due to a difference between the measurement standard temperature and an actual temperature of water as hot water or cold water is discharged to the water purifier. Thus, the error caused by a temperature difference may be resolved, and an accurate measurement value may be obtained.

Additionally, the water purifier and the control method thereof according to the present disclosure may involve cooling the sensing device rapidly with room-temperature water when the sensing device is heated and has a temperature greater than the measurement standard temperature after hot water is discharged. Thus, the temperature of a cooling device may return to a temperature that is the same as or similar to the measurement standard temperature rapidly, and an accurate measurement value of water quality may be obtained rapidly.

Further, the water purifier and the control method thereof according to the present disclosure may involve closing the flow path of the sensing device and increasing the temperature of the sensing device naturally, when the sensing device cools and has a temperature less than the measurement standard temperature after cold water is discharged. Thus, the temperature of the cooling device may return to a temperature the same as or similar to the measurement standard temperature such that an accurate measurement value of water quality is obtained, and the sensing device may not be forcibly heated to increase its temperature, thereby ensuring energy savings.

It will be understood that when an element or layer is referred to as being “on” another element or layer, the element or layer can be directly on another element or layer or intervening elements or layers. In contrast, when an element is referred to as being “directly on” another element or layer, there are no intervening elements or layers present. As used herein, the term “and/or” includes any and all combinations of one or more of the associated listed items.

It will be understood that, although the terms first, second, third, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section could be termed a second element, component, region, layer or section without departing from the teachings of the present invention.

Spatially relative terms, such as “lower”, “upper” and the like, may be used herein for ease of description to describe the relationship of one element or feature to another element(s) or feature(s) as illustrated in the figures. It will be understood that the spatially relative terms are intended to encompass different orientations of the device in use or operation, in addition to the orientation depicted in the figures. For example, if the device in the figures is turned over, elements described as “lower” relative to other elements or features would then be oriented “upper” relative to the other elements or features. Thus, the exemplary term “lower” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the disclosure are described herein with reference to cross-section illustrations that are schematic illustrations of idealized embodiments (and intermediate structures) of the disclosure. As such, variations from the shapes of the illustrations as a result, for example, of manufacturing techniques and/or tolerances, are to be expected. Thus, embodiments of the disclosure should not be construed as limited to the particular shapes of regions illustrated herein but are to include deviations in shapes that result, for example, from manufacturing.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Any reference in this specification to “one embodiment,” “an embodiment,” “example embodiment,” etc., means that a particular feature, structure, or characteristic described in connection with the embodiment is included in at least one embodiment of the invention. The appearances of such phrases in various places in the specification are not necessarily all referring to the same embodiment. Further, when a particular feature, structure, or characteristic is described in connection with any embodiment, it is submitted that it is within the purview of one skilled in the art to effect such feature, structure, or characteristic in connection with other ones of the embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. A water purifier, comprising:
a heater configured to heat water;
a cooler configured to cool water;
a sensing assembly that is positioned to receive water discharged from the heater and the cooler;
at least one valve to control a flow of water in the water purifier; and
a tap that is fluidly connected to the sensing assembly and from which water is discharged,
wherein when heated water from the heater is discharged from the tap,
the at least one valve causes, following a set minimum standby period after discharge of the heated water is completed, water to flow to the sensing assembly such that a temperature at a measurement point of the sensing assembly becomes a first set temperature, and
after the temperature at the measurement point of the sensing assembly becomes the first set temperature, the sensing assembly measures water quality.

2. The water purifier of claim 1, wherein when cooled water from the cooler is discharged from the tap,
the at least one valve prevents, following a set minimum standby period after discharge of the cooled water is completed, water from flowing to the sensing assembly until the temperature at the measurement point of the sensing assembly becomes a second set temperature, and
after the temperature at the measurement point of the sensor becomes the second set temperature, the at least one valve allows water to flow to the sensing assembly, and the sensing assembly measures water quality of water flowing to the sensing assembly.

3. The water purifier of claim 2, wherein the sensing assembly comprises:
a body having a space in which water flows;
a first sensor disposed at the body and configured to measure a temperature of water flowing in the body; and
a second sensor disposed at the body to be separate from the first sensor and configured to measure water quality of water flowing in the body.

4. The water purifier of claim 3, wherein the second sensor comprises a TDS sensor configured to sense an amount of total dissolved solids (TDS) included in water flowing in the body.

5. The water purifier of claim 2, wherein the first set temperature matches the second set temperature.

6. The water purifier of claim 2, wherein the first set temperature is greater than the second set temperature.

7. The water purifier of claim 6, wherein the first set temperature is greater than a measurement standard temperature for the sensing assembly, and the second set temperature is less than the measurement standard temperature for the sensing assembly.

8. The water purifier of claim 2, wherein
the water purifier comprises a drain disposed between the sensing assembly and the tap, and configured to drain water outward from the water purifier without passing through the tap, and
water that is flowing to the sensing assembly after discharge of the heated water or the cooled water is drawn into the drain and drained outward from the water purifier without passing through the tap.

9. The water purifier of claim 8, wherein after discharge of the heated water from the tap, and the temperature at the measurement point of the sensing assembly recaching the first set temperature, the sensing assembly measures water quality of water that flows in the sensing assembly and is drained outward through the drain.

10. The water purifier of claim 8, wherein after discharge of cooled water from the tap and the temperature at the measurement point of the sensing assembly reaching the second set temperature, the sensing assembly measures water quality of water that flows in the sensing assembly, and is drained outward through the drain.

11. The water purifier of claim 8, wherein the at least one valve includes a first diverter valve that directs water to one of the heater or the cooler; and a second diverter valve that directs water to one of the drain or the tap.

12. The water purifier of claim 2, wherein while the sensing assembly measures water quality, the heater and the cooler are inactive so room-temperature water is flowing to the sensing assembly.

13. The water purifier of claim 12, wherein, after discharge of the heated water is completed, the heater and the cooler are inactive such that room-temperature water flows to the sensing assembly while the temperature at the measurement point of the sensing assembly is changing to the first set temperature.

14. The water purifier of claim 1, further comprising a filter that purifies water provided to at least one of the heater or the cooler.

15. A control method of a water purifier, the control method comprising:
discharging heated water, a temperature at a measurement point of a sensor of the water purifier being increased during discharging of heated water;
after discharging of heated water, allowing room-temperature water to flow to the sensor;
checking when the temperature at the measurement point of the sensor reaches a first set temperature due to room-temperature water flowing to the sensor; and
measuring water quality by the sensor after the temperature at the measurement point of the sensor reaches the first set temperature following discharging of heated water.

16. The control method of claim 15, further comprising:
discharging cooled water, the temperature at the measurement point of the sensor being decreased during discharging of cooled water;
after discharging of cooled water, closing a flow path of the sensor;
determining when the temperature at the measurement point of the sensor reaches a second set temperature after closing the flow path;
after the temperature at the measurement point of the sensor reaches the second set temperature following discharging of cooled water, reopening the flow path to allow room-temperature water into the flow path, and
measuring, by the sensor, water quality of the room-temperature water after the flow path is reopened.

17. The control method of claim 15, further comprising:
draining, via a drain provided downstream of the sensor, room-temperature water flowing to the sensor.

18. The control method of claim 17, wherein as the temperature at a measurement point of the sensor reaches the first set temperature, the sensor measures water quality in a state in which room-temperature water flows in the sensor and is drained outward through the drain part and after heated water is completely discharged from the water purifier.

19. The control method of claim 16, further comprising:
draining, via a drain provided downstream of the sensor, the room-temperature water flowing into the flow path and in which water quality is measured by the sensor.

20. The control method of claim 15, further comprising:
filtering water to be dispensed, wherein at least a portion of filtered water passes through a heater configured to heat water and then through the sensor,
wherein the discharging of heated water includes activating the heater to heat the portion of filtered water passing through the heater, and
wherein allowing room-temperature water to flow to the sensor includes deactivating the heater after such that the portion of filtered water passing through the heater is at room-temperature.
